(19) United States
(12) Patent Application Publication
(10) Pub. No.: US 2014/0163383 A1
(43) Pub. Date: Jun. 12, 2014

(54) Self-Flushing Intravascular Catheter Apparatus and Associated Methods

(71) Applicant: Volcano Corporation, San Diego, CA (US)

(72) Inventors: Dylan Van Hoven, Oceanside, CA (US); Michael Reiter, San Diego, CA (US)

(21) Appl. No.: 14/096,805

(22) Filed: Dec. 4, 2013

Related U.S. Application Data
(60) Provisional application No. 61/733,744, filed on Dec. 5, 2012.
(60) Provisional application No. 61/737,440, filed on Dec. 14, 2012.

(51) Int. Cl. A61B 8/00; A61B 8/12

(52) U.S. Cl. CPC A61B 8/445; A61B 8/12
USPC 600/467

(57) ABSTRACT

An intravascular catheter having an elongated sheath surrounding an elongated flexible inner structure movable relative thereto is provided with various representative embodiments of flushing apparatus carried by the inner structure. Such flushing apparatus is operative in response to movement of the inner structure relative to the sheath to induce a flow of flushing fluid from a source thereof through the interior of the sheath and then discharge the flushing fluid from the sheath. In one embodiment thereof the flushing apparatus includes an impeller structure disposed on the inner structure. In another embodiment thereof the flushing structure includes an annular seal disposed on the inner structure in sliding and sealing engagement with the interior surface of the sheath.

### CROSS-REFERENCE TO RELATED APPLICATIONS

The present application claims the benefit of the filing dates of provisional U.S. patent application No. 61/733,774 filed Dec. 5, 2012 and provisional U.S. patent application No. 61/737,440 filed Dec. 14, 2012. The entire disclosures of these provisional applications are incorporated herein by this reference.

### TECHNICAL FIELD

Embodiments of the present disclosure relate generally to the field of medical devices and, more particularly, to apparatus and methods for flushing catheters used in internal vasculature diagnostic procedures.

### BACKGROUND

Various techniques and systems have recently been developed to visualize the anatomy of vascular occlusions by using intravascular ultrasound (IVUS) imaging. IVUS techniques are catheter-based and provide a real-time sectional image of the arterial lumen and the arterial wall. An IVUS catheter includes one or more ultrasound transducers at the distal tip of the catheter by which images containing cross-sectional information of the artery under investigation can be determined. IVUS imaging permits visualization of the configuration of the obstructing material and, in varying degrees, the boundaries of the intimal and medial layers of the arterial wall.

One common type of IVUS imaging catheter system typically includes an arrangement in which a single transducer at the distal end of the catheter is rotated at high speed (up to about 2000 rpm) to generate a rapid series of 360-degree ultra sound sweeps. Such speeds result in generation of up to about thirty images per second, effectively presenting a real-time image of the diseased artery.

The transducer is mounted on the end of a drive shaft or cable that is connected to a motor drive at the proximal end of the catheter. The rotating transducer is housed within a sheath that does not interfere with the ultrasound and protects the artery from the rapidly spinning drive shaft. Thus, an IVUS imaging (or “sensing”) catheter may be advanced to the region of an occlusion using conventional angiographic techniques and then may be operated to provide real-time sectional images of the vascular lumen in the arterial wall, including the occluding material and intimal and medial layers of the artery wall.

Other types of catheter-based systems for use in visualizing the internal anatomy of body portions implementing sheath-enclosed movable sensing/imaging element disposed on elongated drive shaft or cable structures are also known, including photo-acoustic, optical coherence tomography, phased array/multiple transducer, and spectroscopic systems. The performance of each of the above-mentioned catheter-based systems may be adversely affected by the presence of air within the interior of its sheath portion adjacent the movable sensing element(s) therein. Air bubbles may be present in the sheath prior to the operation of the system, or may be generated during system operation by rotation of the flexible drive shaft or cable disposed proximally of the sensing/imaging element(s). The presence of these air bubbles tends to undesirably corrupt the image or data which the system is designed to generate.

To rid the particular system of air within the sheath portion of the overall catheter structure, a flushing operation is typically performed. As conventionally implemented, such internal air-removal operation is carried out by forcing a flushing liquid, such as a saline solution, distally through the entire length of the sheath for discharge out its open distal end. This previously utilized flushing technique carries with it various known problems, limitations and disadvantages.

For example, because of the significant length and very small internal diameter of the sheath (typically in the range of from about 0.3 cm to about 20 cm in useable length and in the range of from about 0.05 cm to about 0.1 cm in internal diameter), forcing flushing liquid from the proximal end of the sheath distally through its entire length requires significant pressure. It also requires additional parts in the form of an attached one-way check valve and a detachable syringe and hose apparatus, thereby undesirably adding to the cost of the overall system. The check valve also creates an obstruction near the hand piece portion of the system when the sensing/imaging structure is manipulated manually. Furthermore, there is currently no preventive measure for distally blocking air bubbles, released from the inside of the drive cable during system operation, from reaching the sensing/imaging structure and adversely affecting its performance.

As can be readily be seen from the foregoing, a need exists for improved apparatus and associated methods for flushing medical catheters of the types generally described above. It is to this need that the present invention is primarily directed.

### SUMMARY

In carrying out principles of the present invention in accordance with a first representatively disclosed embodiment thereof, a medical sensing catheter apparatus is provided which is illustratively an IVUS catheter, but could alternatively be another type of medical sensing catheter such as a photo-acoustic, optical coherence tomography, phased array/multiple transducer, or spectroscopic type of catheter.

The catheter structure provides for improved flushing thereof, and illustratively includes an elongated flexible sheath having a proximal end, and an open distal end insertable into a body area of a patient, and an elongated flexible inner structure longitudinally extending through the interior of the sheath and being movable relative thereto. The inner structure has a distal end portion with a sensing element disposed thereon and operative to generate signals useable to create diagnostic information with respect to the patient body area, representatively an artery. Illustratively, the sensing element is an ultrasound transducer.

Disposed between the inner structure and the sheath is a substantially barrier free space or passage that circumscribes the inner structure and extends along substantially the entire length of the inner structure received in the sheath. According to a key feature of the invention, an impeller structure is carried by the inner structure and is operative, in response to rotation of the inner structure relative to the sheath, to flow a flushing liquid from a source thereof proximally through the sheath toward the support structure, along the exterior of the support structure, and then through the substantially barrier free space, in a manner preventing air from being interposed between the support structure and a facing interior side surface portion of the sheath. In representatively disclosed embodiments thereof the impeller structure may be external impeller vanes or a spiraling surface groove externally formed on the distal end portion of the inner structure.

When the catheter is representatively disposed in an artery, blood from within the artery is used as the flushing liquid, and may subsequently be returned to the artery from the substantially barrier free space via an optional blood outlet port formed in the sheath. Alternatively, prior to use of the catheter within the body, the interior of the sheath may be flushed in the same manner using a non-blood flushing liquid such as a saline solution.

According to another disclosed aspect of the invention, a method is provided for flushing air from within a medical sensing catheter generally having a construction as described above, the method comprising the steps of (1) configuring the distal end portion of the inner structure to draw a flushing liquid from a source thereof inwardly through the open distal end of the sheath in response to rotation of the distal end portion of the inner structure relative to the sheath; and (2) causing flushing liquid from a source thereof to sequentially flow proximally through the sheath toward the distal end portion of the inner structure, along the exterior of the distal end portion of the inner structure, and then proximally through the substantially barrier free space, in a manner preventing air from being interposed between the distal end portion of said inner structure and a facing interior side surface portion of the sheath, by rotating the inner structure relative to said sheath. In the catheter flushing technique provided by this exemplary method, either blood or a non-blood flushing liquid may be utilized.

Using the representatively disclosed catheter construction and associated catheter use as provided by the present invention, the proximally driven flow of flushing liquid through the catheter sheath continuously prevents internal sheath air adjacent the support structure from being undesirably interposed between the transducer and a facing interior side surface portion of the sheath, and also forms a moving liquid barrier that prevents air generated by the rotating inner structure from traveling distally to the transducer carrying support structure.

In a second representatively disclosed embodiment of the present invention, a medical sensing catheter apparatus is provided which is illustratively an IVUS catheter, but could alternatively be another type of medical sensing catheter such as a photo-acoustic, optical coherence tomography, phased array/multiple transducer, or spectroscopic type of catheter. The catheter structure provides for improved flushing thereof and illustratively includes an elongated flexible sheath having a proximal end, and an open distal end insertable into a body area of a patient, and an elongated flexible inner structure longitudinally extending through the interior of the sheath and being movable relative thereto, the inner structure having a distal end portion with a sensing element disposed thereon and operative to generate signals useable to create diagnostic information with respect to the patient body area. According to a key feature of the catheter apparatus, an annular seal structure is coaxially and externally carried by the inner structure proximally of the sensing element, the seal structure slidingly and sealingly engaging the interior surface of the sheath and being movable with the inner structure relative to the sheath.

According to a structural aspect of the catheter apparatus, the annular seal structure permits a distal end portion of the sheath to be flushed in a manner expelling undesirable air therefrom by proximally moving the inner structure relative to the sheath to draw flushing liquid proximally into the sheath through its distal end, and then moving the inner structure distally relative to the sheath to discharge the received fluid, and air therein, outwardly through the distal sheath end to thereby obviate the previous necessity of performing the necessary flushing operation by forcing flushing fluid distally through the entire length of the sheath.

In an alternate configuration of the second embodiment of the present invention, an impeller structure is provided on the portion of the inner structure that carries the sensing element and is operative to draw flushing liquid proximally through a distal end portion of the sheath, towards the seal structure, in response to rotation of the inner structure relative to the sheath, to flush a distal tip portion of the sheath without translating the inner structure relative to the sheath.

In accordance with another disclosed aspect of the present invention, an air flushing method is provided for use with a medical sensing catheter having an elongated flexible sheath with a proximal end and an open distal end insertable into a body area of a patient, and an elongated flexible inner structure longitudinally extending through the interior of the sheath and being movable relative thereto, the inner structure having a distal end portion with a sensing element disposed thereon and operative, during movement of said inner structure relative to said sheath, to generate signals useable to create diagnostic information with respect to the patient body area.

From a broad perspective, the method comprises the steps of flowing a quantity of flushing fluid inwardly through the open distal end of said sheath toward the sensing element, and then discharging at least a portion of the quantity of flushing fluid, and air previously disposed within the interior of the sheath, outwardly through a distal end portion of the sheath. According to disclosed features of the method, the flowing and discharging steps may respectively include the step of proximally and distally translating the inner structure through the sheath, or may each be performed by rotating the inner structure relative to the sheath.

Catheter apparatus and associated flushing methods representatively disclosed herein provide substantial improvements in the overall catheter flushing operation. For example, when blood is utilized as a flushing liquid the necessity for the use of a secondary acoustic media is eliminated, thereby desirably lessening the total material cost for the diagnostic procedure, and also reduces the quantity of particulates that may potentially be introduced into the body using a distally directed flushing procedure. Catheter structure and associated methods representatively disclosed herein further improve work flow and ease of catheter use for the operator, reduce the time needed for the flushing procedure, and improve diagnostic image quality via the elimination of air-related image artifacts.

### BRIEF DESCRIPTION OF THE DRAWINGS

FIG. 1 is a highly schematic partially cross-sectioned view of representative medical sensing catheter apparatus embodying principles of the present invention;

FIG. 2 is a side elevational view of an alternate embodiment of a transducer support portion of the FIG. 1 catheter apparatus;

FIG. 3 is a partially cutaway schematic elevational view of a second embodiment of the FIG. 1 medical sensing catheter apparatus;

FIG. 4 is a cross-sectional enlargement of the dashed circle area “4” in FIG. 3;

FIGS. 5A-5D sequentially depict, in schematic form, a representative catheter flushing method embodying principles of the present invention;

FIG. 6 is an enlarged scale schematic cross-sectional view of a distal end portion of an alternate configuration of the catheter apparatus of FIG. 5D being flushed by an associated alternate flushing method embodying principles of the present invention; and

FIG. 7 illustrates an alternate configuration of the distal end portion of the inner catheter structure shown in FIG. 6.

### DETAILED DESCRIPTION

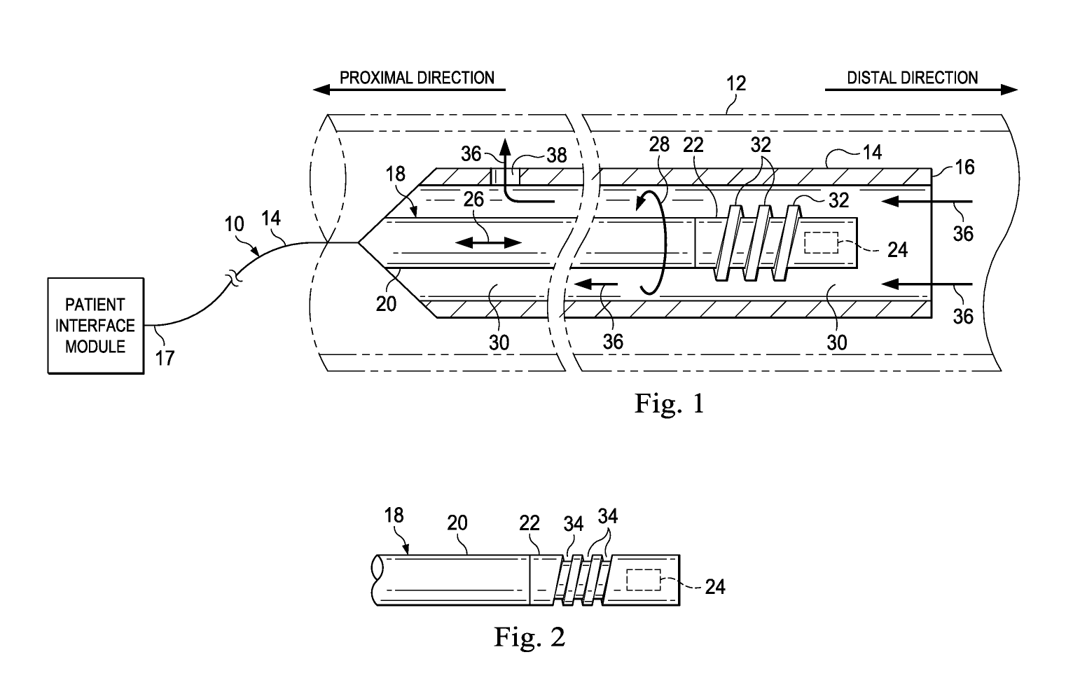

Schematically depicted in FIG. 1 is medical sensing catheter apparatus which is representatively in the form of an intravascular catheter 10 that may be operatively inserted in an artery 12 of a patient to perform diagnostic artery imaging procedures. By way of non-limiting example, the catheter 10 is an IVUS (Intravascular Ultrasound) catheter of the single rotatable transducer type, but could alternatively be one of a variety of other types of catheter-based systems for use in visualizing the internal anatomy of body portions implementing sheath-enclosed movable sensing/imaging elements disposed on elongated drive shaft or cable structures, including but not limited to photo-acoustic, optical coherence tomography, and spectroscopic systems, without departing from principles of the present invention.

Catheter 10 includes an elongated flexible tubular outer sheath 14 having an open distal end 16 and a proximal end 17. The useable length of the sheath 14 (i.e., the length thereof operatively insertable into a patient) is greatly larger than its internal diameter. Illustratively, the useable sheath length is in the range of from about 0.3 cm to about 20 cm, and the internal sheath diameter is in the range of from about 0.05 cm to about 0.1 cm. Movably disposed within the interior of the sheath 14 is an elongated flexible inner structure 18 comprising an elongated flexible drive shaft or cable 20 with a support structure 22 (typically referred to as a “can”) secured to its distal end and carrying a sensing/imaging element illustratively in the form of a single ultrasound transducer 24.

The proximal end 17 of the sheath, and the proximal end of the drive shaft or cable 20 therein, are operatively connected to a suitable PIM (patient interface module), of conventional construction, which provides electrical power to the transducer 24 and receives imaging signals therefrom. The PIM may also be operative to selectively drive the inner structure 18 translationally and rotationally relative to the sheath 14 as respectively illustrated by the arrows 26,28 in FIG. 1. Alternatively, a separate drive structure may similarly drive the inner structure 18 relative to the sheath 14, or such translational and rotational movement of the inner structure 18 relative to the sheath 14 may be manually achieved if desired.

As can be seen in FIG. 1, disposed between the outer sheath 14 and the inner structure 18 is a substantially barrier-free space or passage 30 that circumscribes the inner structure 18 and extends along substantially the entire length of the inner structure 18 received in the sheath 14. According to a feature of the present invention, an impeller structure is externally disposed on the support structure 22 and is illustratively in the form of a plurality of impeller vanes 32 disposed on the support structure portion 22 of the flexible inner structure 18 proximally of the transducer 24. Alternatively, as shown in FIG. 2, the impeller structure could comprise a suitably spiraled groove 34 formed in the outer side surface of the support structure 22 proximally of the transducer 24.

With the catheter 10 received in the artery 12 as shown in FIG. 1, the interior of the sheath 14 may be easily flushed, to expel undesirable air therein, by simply rotating the inner structure 18 relative to the sheath 14 as indicated by the arrow 28. Such rotation, by virtue of the impeller structure 32 (or the FIG. 2 impeller structure 34 as the case may be) causes blood 36 from within the artery 12 to be flowed inwardly through the open distal end 16 of the sheath 14, exteriorly along the support structure 22, and then proximally through the substantially barrier-free annular passage 30. The proximally flowing blood 36 is thus utilized as a flushing liquid to rid the interior of the sheath 14 of undesirable air and may be transferred back into the artery 12 via an optional side wall blood exit port 38 formed in the sheath 14.

This proximally driven flow of flushing liquid through the sheath 14 continuously prevents internal sheath air adjacent the support structure 22 from being undesirably interposed between the transducer 24 and a facing interior side surface portion of the sheath 14, and also forms a moving liquid barrier that prevents air generated by the rapidly spinning drive shaft or cable 20 from traveling distally to the support structure 22.

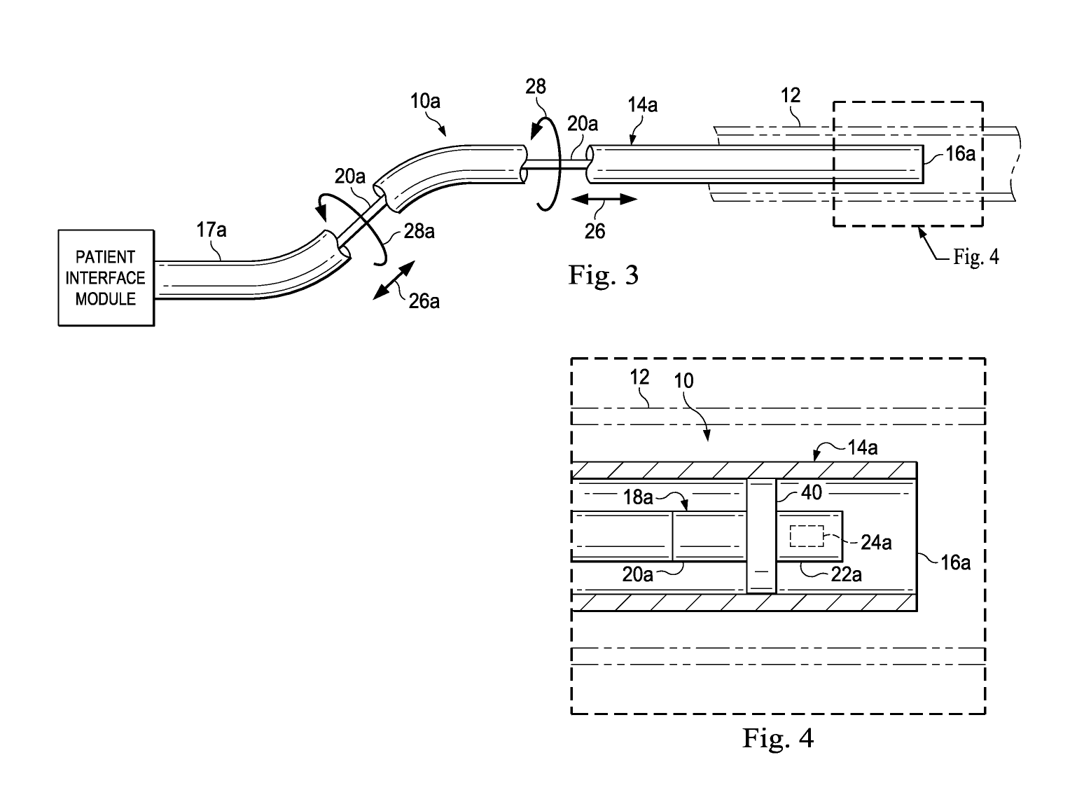

Schematically depicted in FIGS. 3 and 4 is an alternate embodiment 10a of the previously described medical sensing catheter apparatus 10. Catheter 10a may be operatively inserted in artery 12 of a patient to perform diagnostic artery imaging procedures. By way of non-limiting example, the catheter 10a is an IVUS (Intravascular Ultra Sound) catheter of the single rotatable transducer type, but could alternatively be one of a variety of other types of catheter-based systems for use in visualizing the internal anatomy of body portions implementing sheath-enclosed movable sensing/imaging elements disposed on elongated drive shaft structures, including photo-acoustic, optical coherence tomography, phased array/multiple transducer, and spectroscopic systems, without departing from principles of the present invention.

Catheter 10a includes an elongated flexible tubular outer sheath 14a having an open distal end 16a and a proximal end 17a. The useable length of the sheath 14a (i.e., the length thereof operatively insertable into a patient) is greatly larger than its internal diameter. Illustratively, the useable sheath length is in the range of from about 0.3 cm to about 20 cm, and the internal sheath diameter is in the range of from about 0.05 cm to about 0.1 cm. Movably disposed within the interior of the sheath 14a is an elongated flexible inner structure 18a comprising an elongated flexible drive shaft or cable 20a with a housing structure 22a secured to its distal end and carrying a sensing element illustratively in the form of a single ultrasound transducer 24a.

The proximal end 17a of the sheath 14a, and the proximal end of the drive cable 20a therein, are operatively connected to a suitable PIM (patient interface module), of conventional construction, which provides electrical power to the transducer 24a and receives imaging signals therefrom. The PIM may also be operative to selectively drive the inner structure 18a translationally and rotationally relative to the sheath 14a as respectively illustrated by the arrows 26a,28a in FIG. 3. Alternatively, a separate drive structure may similarly drive the inner structure 18a relative to the sheath 14a, or such translational and rotational movement of the inner structure 18a relative to the sheath 14a may be manually achieved if desired.

Referring now to FIG. 4, according to a key aspect of the present invention, the catheter 10a also comprises an annular seal structure 40 which coaxially circumscribes and is anchored to the housing portion 22a of the inner structure 18a proximally adjacent the transducer 24a. The seal structure 40 slidably and sealingly engages the interior surface of the sheath 14a and is translationally and rotationally movable with the inner structure 18a relative to the sheath 14a. By way of non-limiting example, the seal structure 40 may be of various types including one or more metal rings (illustratively welded stainless steel rings), a tape material such as a PTFE tape material, or at least one annular resilient seal member overmolded onto the inner structure 18.

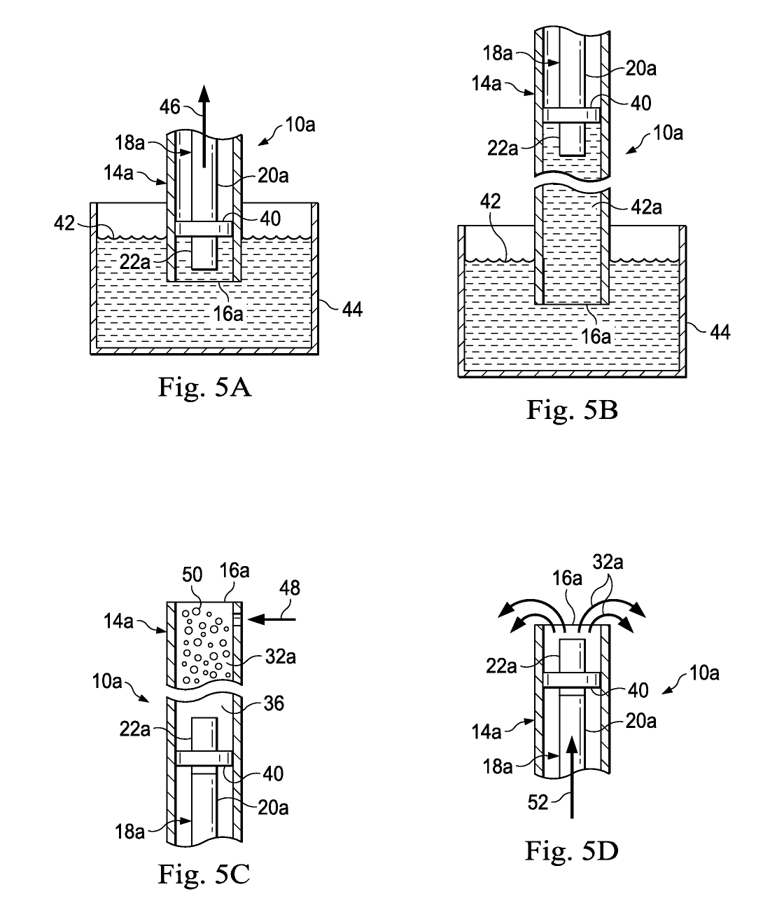

The unique provision of the seal structure 40 permits the sheath portion 14a of the catheter 10a to be flushed, to remove air from its interior, without the previous problems, limitations and disadvantages associated with forcing a flushing liquid distally through the entire length of the interior of the sheath 14a. An example of how such improved flushing may be achieved via the present invention is sequentially depicted in schematic form in FIGS. 5A-5D.

First, as shown in FIG. 5A, with the housing structure 22a adjacent the downwardly facing open distal end 16a of the sheath 14a, the distal sheath end 16a is dipped into a quantity of flushing liquid, representatively a saline solution 42, within a suitable container 44. The inner structure 18a is then raised within the sheath 14a (as indicated by the arrow 46 in FIG. 5A) to its FIG. 5B position. This raising of the seal structure 40 creates a vacuum in the interior portion of the sheath 14a below it, thereby drawing a quantity 42a of the saline solution 42 upwardly into the interior portion of the sheath 14a below the seal structure 40.

Next, as shown in FIG. 5C, the distal sheath end 16a is inverted to face upwardly, and the sheath 14 is tapped (as indicated by the arrow 48 in FIG. 5C) to cause air 50 within the sheath 14a distally of the seal structure 40 to rise to the distal sheath end 16a. Finally (as indicated by the arrow 52 in FIG. 5D), the inner structure 18a is forced upwardly toward the distal sheath end 16a to thereby discharge saline solution 42a (with the air 50 entrained therein) outwardly through the open distal sheath end 16a, thereby completing the catheter sheath flushing operation.

It should be noted that using this improved flushing technique requires that only a distal tip end portion of the overall sheath 14a need be flushed to ready the catheter 10a for patient use, and that the overall flushing operation is greatly simplified and quickened. Further, the equipment cost to achieve the necessary flushing is desirably reduced. The efficiency of the flushing operation is also enhanced due to the fact that the seal structure 40 forms a barrier against air bubbles, generated by the rapid operational rotation of the flexible drive cable 20a, distally reaching and interfering with the imaging performance of the transducer 24a.

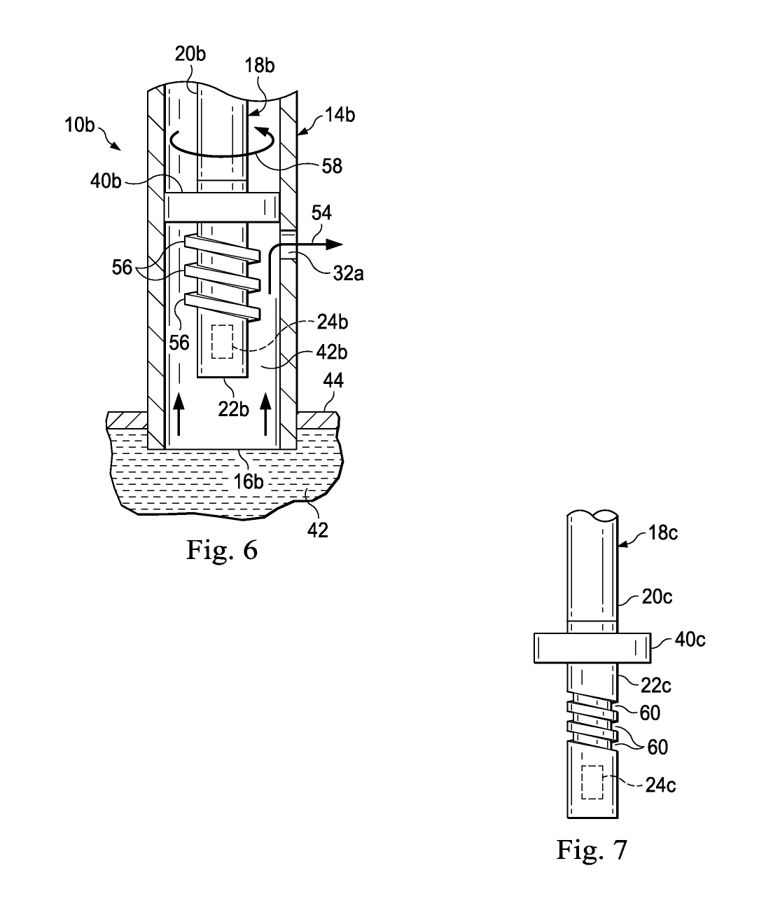

FIG. 6 schematically depicts an alternate flushing method carried out using, in an alternate catheter embodiment 10b, modified sheath and housing structures 14b,22b embodying principles of the present invention. The sheath 14b shown in FIG. 6 is modified by forming a side wall vent opening 54 therein adjacent its open distal end 16b. The housing 22b shown in FIG. 6 is modified by forming externally thereon an impeller structure, representatively a plurality of impeller vanes 56, disposed between the transducer 24b and the seal structure 40b.

With the distal end 16b of the sheath 14b dipped into the saline solution 42, the housing structure 22b adjacent the distal end 16b, and the seal structure 40 disposed proximally of the vent opening 54, the inner structure 18b is rotationally driven, as indicated by the arrow 58, without translating the inner structure 18b relative to the sheath 14b. The rotating impeller structure 56 upwardly draws a portion 42b of the saline solution 42 into the interior of the sheath 24b and discharges it (with air from below the seal structure 40) outwardly through the vent opening 54 to complete the flushing operation.

FIG. 7 schematically depicts an alternate embodiment 22c of the housing structure 22b shown in FIG. 6. The FIG. 7 housing structure 22c is identical to the FIG. 6 housing structure 22b with the exception that the impeller structure externally formed on the FIG. 6 housing structure 22c (which performs the same function as the vanes 56 in FIG. 6) is defined by a spiraling exterior side wall groove 60 formed in the outer surface of the housing structure 22c and disposed between the transducer 24c and the annular seal structure 40c.

As can be seen, the present invention in the illustrative embodiments thereof described above provides substantial improvements in the overall catheter flushing operation. For example, when blood is utilized as a flushing liquid the necessity for the use of a secondary acoustic media is eliminated, thereby desirably lessening the total material cost for the diagnostic procedure, and also reduces the quantity of particulates that may potentially be introduced into the body using a distally directed flushing procedure. The above-described flushing structure and method further improve work flow and ease of use for the operator, reduce the time needed for the flushing procedure, and improve diagnostic image quality via the elimination of air-related image artifacts.

The foregoing detailed description is to be clearly understood as being given by of illustration and example only, the spirit and scope of the present invention being limited solely by the appended claims.

What is claimed is:

1. Medical sensing catheter apparatus comprising:
an elongated flexible sheath having a proximal end, and an open distal end insertable into a body area of a patient;
an elongated flexible inner structure longitudinally extending through the interior of said sheath and being movable relative to said sheath, said inner structure including an elongated flexible drive member having a distal end, a support structure secured to said distal end of said drive member, and a sensing element carried by said support structure and operative to generate signals useable to create diagnostic information with respect to the patient body area; and
flushing apparatus carried by said inner structure and being operative in response to movement of said inner structure relative to said sheath to induce a flow of flushing fluid from a source thereof through the interior of said sheath and then discharge the flushing fluid from said sheath.

2. The medical sensing catheter apparatus of claim 1 wherein:
said inner structure and said sheath having disposed therebetween a substantially barrier free space circumscribing said inner structure and extending along substantially the entire length of said inner structure received in said sheath, and
said flushing apparatus comprises an impeller structure carried by said inner structure and being operative, in response to rotation of said inner structure relative to said sheath to flow the flushing fluid proximally through said sheath toward said support structure, along the exterior of said support structure, and then through a portion of said substantially barrier free space, in a manner inhibiting air from being interposed between said support structure and a facing interior side surface portion of said sheath.

3. The medical sensing catheter apparatus of claim 2 wherein:
said impeller structure is disposed on said support structure.

4. The medical sensing catheter apparatus of claim 2 wherein:
said impeller structure comprises a plurality of impeller vanes.

5. The medical sensing catheter apparatus of claim 2 wherein:
said impeller structure comprises a spiraling groove externally disposed on said support structure.

6. The medical sensing catheter apparatus of claim 2 wherein:
said sensing element is an ultrasound transducer.

7. The medical sensing catheter apparatus of claim 2 wherein:
a side wall liquid outlet port formed in said sheath and being proximally spaced apart from said support structure.

8. The medical sensing catheter apparatus of claim 2 wherein:
said medical sensing catheter apparatus is an IVUS catheter.

9. The medical sensing catheter apparatus of claim 2 wherein:
said medical sensing catheter apparatus is sized for use in a human artery.

10. The medical sensing catheter apparatus of claim 2 wherein:
said sheath has a useable length of within the range of from about 0.3 cm to about 20 cm, and an internal diameter within the range of from about 0.05 cm to about 0.1 cm.

11. The medical sensing catheter apparatus of claim 1 wherein:
said flushing apparatus comprises an annular seal structure coaxially and externally carried by said inner structure proximally of said sensing element, said annular seal structure slidingly and sealingly engaging the interior surface of said sheath and being movable with said inner structure relative to said sheath.

12. The medical sensing catheter apparatus of claim 11 wherein:
said sensing element includes an ultrasound transmitter.

13. The medical sensing catheter apparatus of claim 11 wherein:
said seal structure includes at least one metallic ring member.

14. The medical sensing catheter apparatus of claim 13 wherein:
said at least one metallic ring member is of a welded stainless steel material.

15. The medical sensing catheter apparatus of claim 11 wherein:
said seal structure includes at least one seal member overmolded onto said inner structure.

16. The medical sensing catheter apparatus of claim 11 wherein:
said seal structure includes a tape material wrapped around said inner structure.

17. The medical sensing catheter apparatus of claim 16 wherein:
said tape material is a PTFE tape material.

18. The medical sensing catheter apparatus of claim 11 wherein:
said medical sensing catheter apparatus comprises an IVUS catheter.

19. The medical sensing catheter apparatus of claim 11 wherein:
said annular seal structure is configured and operative to facilitate expulsion of air from a portion of the interior of said sheath extending distally from said seal structure by sequentially drawing a flushing fluid distally into said sheath by proximally moving said inner structure relative thereto, causing the air to be positioned in the drawn in flushing fluid adjacent said open distal end of said sheath, and then distally moving the inner structure relative to said sheath to expel air through the distal end thereof.

20. The medical sensing catheter apparatus of claim 11 further comprising:
an impeller structure carried on said inner structure distally of said seal structure and operative, during rotation of said inner structure relative to said sheath, to draw fluid through said sheath proximally toward said seal structure.

21. The medical sensing catheter apparatus of claim 20 wherein:
said impeller structure comprises a spiraling vane structure externally disposed on said inner structure.

22. The medical sensing catheter apparatus of claim 20 wherein:
said impeller structure comprises a spiraling external groove formed on said inner structure.

23. The medical sensing catheter apparatus of claim 20 wherein:
said medical sensing catheter apparatus further comprises a vent opening extending through said sheath and positioned to form an outlet for fluid drawn through said sheath proximally toward said seal structure by said impeller structure,
whereby a distal portion of said sheath may be flushed by immersing a distal end portion of said sheath into a flushing fluid and rotating said inner structure relative to said sheath.

24. The medical sensing catheter apparatus of claim 11 wherein:
said sheath has a useable length of within the range of from about 0.3 cm to about 20 cm, and an internal diameter within the range of from about 0.05 cm to about 0.1 cm.

25. A method of flushing air from within a medical sensing catheter having an elongated flexible sheath with a proximal end and an open distal end insertable into a body area of a patient, and an elongated flexible inner structure longitudinally extending through the interior of said sheath and being movable relative thereto, said inner structure having a distal end portion with a sensing element disposed thereon and operative to generate signals useable to create diagnostic information with respect to the patient body area, said inner structure and said sheath having disposed therebetween a substantially barrier free space circumscribing said inner structure and extending along substantially the entire length of said inner structure received in said sheath, said method comprising the steps of:
configuring said distal end portion of said inner structure to draw a flushing liquid from a source thereof inwardly through said open distal end of said sheath in response to rotation of said distal end portion of said inner structure relative to said sheath; and
rotating said inner structure relative to said sheath, thereby causing flushing liquid from a source thereof to sequentially flow proximally through said sheath toward said distal end portion of said inner structure, along the exterior of said distal end portion of said inner structure, and then proximally through a portion of said substantially barrier free space, in a manner inhibiting air from being interposed between said distal end portion of said inner structure and a facing interior side surface portion of said sheath.

26. The method of claim 25 wherein:
said configuring step includes the step of securing an impeller vane structure to said distal end portion of said inner structure.

27. The method of claim 25 wherein:
said configuring step includes the step of forming a spiraling groove on an external surface portion of said distal end portion of said inner structure.

28. The method of claim 25 wherein:
said method further comprises the step of inserting said medical sensing catheter into an artery of a patient, and
said causing step is performed using blood from within the artery as said flushing liquid.

29. The method of claim 28 further comprising the step of:
returning the blood to the artery from said substantially barrier free space.

30. The method of claim 29 wherein:
said method further comprises the step of forming a side wall blood exit port in said sheath in a proximally spaced relationship with said distal end portion of said inner structure, and said returning step is performed by flowing blood outwardly through said blood exit port.

31. The method of claim 25 wherein:
said causing step is performed using a non-blood flushing liquid.

32. The method of claim 31 wherein:
said causing step is performed using a saline solution.

33. A method of flushing air from within a medical sensing catheter having an elongated flexible sheath with a proximal end and an open distal end insertable into a body area of a patient, and an elongated flexible inner structure longitudinally extending through the interior of said sheath and being movable relative thereto, said inner structure having a distal end portion with a sensing element disposed thereon and operative, during movement of said inner structure relative to said sheath, to generate signals useable to create diagnostic information with respect to the patient body area, said method comprising the steps of:
flowing a quantity of flushing fluid inwardly through said open distal end of said sheath toward said sensing element, and then
discharging at least a portion of the quantity of flushing fluid, and air previously disposed within the interior of said sheath, outwardly through a distal end portion of said sheath.

34. The method of claim 33 wherein:
said flowing step includes the step of proximally translating said inner structure through said sheath.

35. The method of claim 33 wherein:
said discharging step is performed in response to distally translating said inner structure through said sheath.

36. The method of claim 33 wherein:
said flowing step includes the step of rotating said inner structure relative to said sheath.

37. The method of claim 33 wherein:
said discharging step includes the step of rotating said inner structure relative to said sheath.

38. The method of claim 33 further comprising the step of:
blocking flushing fluid flow proximally past a distal end portion of said inner structure using an annular seal structure secured to and circumscribing said inner structure proximally adjacent said sensing element.

39. The method of claim 38 further comprising the steps of forming a sidewall vent hole through said sheath, and
positioning said seal structure proximally of said vent hole.

40. The method of claim 33 wherein:
at least one of said flowing and discharging steps is performed in response to rotation of said inner structure relative to said sheath using an impeller structure externally disposed on a distal end portion of said inner structure.
